(19) United States
(12) Patent Application Publication
(10) Pub. No.: US 2012/0088726 A1
(43) Pub. Date: Apr. 12, 2012

(54) MUCOADHESIVE XYLOGLUCAN-CONTAINING FORMULATIONS USEFUL IN MEDICAL DEVICES AND IN PHARMACEUTICAL FROMULATIONS

(75) Inventors: Giuseppe Bottoni, Bologna (IT); Paola Maffei, Bologna (IT); Annalisa Sforzini, Bologna (IT); Mascia Federici, Bologna (IT); Carla Caramella, Bologna (IT); Silvia Rossi, Bologna (IT); Giuseppe Claudio Viscomi, Bologna (IT)

(73) Assignee: ALFA WASSERMANN, S.P.A., Bologna (IT)

(21) Appl. No.: 13/277,130

(22) Filed: Oct. 19, 2011

Related U.S. Application Data
(62) Division of application No. 11/921,286, filed on Nov. 29, 2007, now abandoned.

(30) Foreign Application Priority Data
Jun. 6, 2005 (IT) BO2005A000388

(51) Int. Cl. A61K 47/36; A61K 31/416; A61K 31/57; A61K 38/23; A61P 31/00; A61P 37/04; A61P 33/02; A61P 31/12; A61P 29/00; A61P 11/14; A61P 11/02; A61K 31/196; A61P 31/10

(52) U.S. Cl. 514/11.9; 514/777; 514/567; 514/407; 514/171

(57) ABSTRACT

Object of the invention are mucoadhesive and controlled release formulations consisting of aqueous solutions containing 0.05% to 5% by weight of a natural purified polymer having xyloglucan structure and 10% to 70% by weight of glycerol. These formulations are suitable for the application on human mucous membranes, such as nasal, oral and vaginal mucous membranes, as moisturizing and softening agents or as pharmaceutical release system. Further objects of the invention are pharmaceutical formulations and medical devices suitable for the application to human mucous membranes, containing the mucoadhesive and controlled release formulations together with active ingredients and excipients.

### FIELD OF THE INVENTION

The present invention relates to mucoadhesive formulations suitable for the application to human mucous membranes, for use as moisturizing and softening agents or as pharmaceutical release systems for local and/or systemic use.

The mucosa is a covering membrane of the internal surface of body cavities and organic channels directly or indirectly connected to the exterior, i.e. oral cavity, gastrointestinal tube, respiratory tract and vaginal cavity. Despite some differences typical of every single apparatus, mucous membranes are essentially constituted of an epithelium covering the cavity of the organ or the channel's lumen and, more deeply, of a connective tissue (tunica propria or mucous derma) with the supporting function; this tissue may be rich in elastic fibre and lymphocytes. Mucous membranes are wetted by the secretion of glands contained in their thickness or directly produced by the glandular epithelium which constitutes them. Such fluids mainly consist of water, glycoproteins (mucin), lipids, mineral salts and enzymes. The basic functions of all secreted fluids are to modulate interactions between epithelial cells and their environment by means of a lubrication/hydratation process, of a control process at chemical-physical and microbiological level of the “ecosystem” cavities acting also as defence barrier against pathogens.

A reduced secretion of fluids causes the inevitable onset of “dryness”, a symptom connected with of a large number of pathological conditions. In fact, mucous dryness is in most cases the first clinical symptom of Sjorgren Syndrome, a systemic autoimmune disease affecting exocrine glands causing the progressive loss of their activity. The clinical picture is characterized by reduction of lachrymal and salivar secretions (ocular dryness with keratoconjunctivitis, xerostomia, dysphagia) and reduction of vaginal mucous and bronchial tree secretions.

Vaginal mucous membrane is constituted of a non-keratinised stratified pavement epithelium. The intermediate layers of the epithelium show glycogen-rich cells. The epithelium lies on an lamina propria without glands and rich in vessels, most of all veins. The mucous membrane under physiological conditions is covered by a lubricating fluid mainly produced by uterine endometrium, endocervical glands (cervical mucus) and main vestibular glands (Bartolino glands) (J. R. Berman et al., Eur. Urol., 38, 20-29, 2000).

Vaginal fluid in fertile women is normally acidic (pH 3.5-4.5), while before puberty as well as after menopause secretions tend to be alkaline. The vaginal fluid is viscous, water represents the major component (90-99%), while mucin (glycoprotein) amounts to 0.5-5% by weight. Mucin is responsible for the chemical-physical characteristics of the vaginal fluid, particularly viscosity and inucoadhesive properties. Vaginal fluid also contains plasmatic proteins, enzymes (protease, betaglucoronidase, acid and alkaline phosphatase and estherase), amino acids, cholesterol, lipids, inorganic ions, lactic acid, acetic acid and desquamated epithelial cells. In addition to vaginal mucus, a serum transudate is also is present in the vagina.

Mucus production and the concentrations of the different components vary during menstrual period (M. M. Garrey, “Ginecologia Illustrate”, Ed. II Pensiero Scientifico, 1974).

The production of vaginal mucus is mainly controlled by sexual steroids which affect the biophysical properties of the secretion. Hormonal alterations can be the cause of a reduction in the mucus production with a consequent reduction of the vaginal mucous lubrication (vaginal dryness). This condition may often turn Lip during menopause when, as a consequence of the reduction of circulating estrogenic levels, genitals undergo anatomical modifications with a consequent change in morphological and functional characteristics. Ovaries become sclerotized, endometrium atrophises, glandular component tends to disappear, cervical channel stenotises and cervical mucus disappears, vagina narrows and loses its elasticity, vaginal walls became thinner, atrophic and dry. The regression is very evident in both vaginal and neck corions and plasmatic transudation disappears.

The reduced production of mucus exposes the mucous membrane to a greater vulnerability versus infections as a consequence of the abrasions that can occur during the sexual intercourse. Such susceptibility can be further increased by vaginal alterations (reduction of intracellular glycogen, modifications of pH and microflora) typical of menopause (G. B. Candiani, V. Danesino, A. Gastaldi, La Clinica Ostetrica e Ginecologica, Ed. Masson, 1992). This condition causes a symptomatology characterized by dyspareunia, burning and itch with not negligible psychological and sexual rebounds.

Transient, periodical or definitve vaginal dryness is a common problem that can appear at any time of life in women, from puberty to menopause and can often make sexual intercourse painful or even impossible.

In addition to menopause, which evidently represents the most common condition causing vaginal dryness, many other causes exist of either endocrine (reduced levels of circulating extrogens) or non-endocrinel nature, such as hysterectomy/ovariectomy, use of oral contraceptives, amenorrhoea, vaginal mucous membrane irritation caused by microbial or chemical agents, treatments with particular drugs, Sjorgren Syndrome, diabetes mellitus and psychic alterations.

As far as oral cavity mucous membrane is concerned, fauces dryness can be the symptom of several clinical pictures related to endocrine pathologies, psychic alterations like anxiety and depression, nutritional deficiencies, cranial nerves lesions; or also the adverse side-effect of several drugs such as anti-depressants, ACE-inhibitors, chemotherapeutics/radiotherapeutics, sympathomi-metic, anti-retroviral, anxiolytic and sedative, anti-arrhythmic, anti-inflammatory, anti-hypertensive, anti-histaminic, anti-ulcer and neuroleptic agents.

A reduction of the secretion also involves manifestations related to an alteration of the protective and regulative system of physiological equilibrium. For example a reduced salivary production can be responsible for oral dryness (xerostomia), taste alteration, increasing of caries onset and gingival inflammation, stomatitis and mastication problems.

Fauces dryness can also he a symptom related to clinical conditions such as subacute or chronic pharyngitis and chronic laryngitis; the etiological agents of these diseases are pathogenic microbial agents and also irritating agents like atmospheric pollutants, tobacco smoke, cold and dry air, chronic irritation caused by gastric acid material (gastroesophageal reflux), (J. Woodley, Bioadhesion, New possibilities for Drug Administrations, Clin. Pharmacokinetic, 40 77-84, 2001).

In the light of the above considerations, it is useful to provide medical devices and pharmaceutical preparations able to reduce or to remove the negative effects directly and/or indirectly related to mucous dryness.

Moreover, since the organs, in which the mucous membranes are present, are also subjected to specific pathologies and moreover they are able to absorb drugs, it is useful to make available medicaments releasing drugs in such sites.

For such purposes bioadhesive polymers, or substances able to intimately adhere to the biological membranes, can be used, for example as disclosed in EP 0770384131 for preparations for the vaginal use.

Xyloglucans are a class of polysaccharides structurally similar to cellulose and intimately associated to it in the cell wall in superior plants. Moreover xyloglucans are one of the main components, most likely acting as an energetic reserve, of plants seeds such as Tamarindus Indica original from India and South-East Asia, Detarium senegalense Gmelin diffused in Africa (in particular in Nigeria), Afzelia africana diffused in Central and East Africa and also in savannas and forests near East Africa coast and Jatoba.

Xyloglucans are characterized by a main chain of (1,4)-β-D-Glucan substituted with side chains of α-D-xylopyranose and β-D-galactopyranosyl-(1,2)-α-D-xylopyranose linked to glycan residues through a α(1,6) bond. The distribution of the residues in the side chains is different in xyloglucans of different types. Three oligomeric units have been identified in the xyloglucans structure, namely monosaccharides, octasaccharides and heptasaccharides which differ from each other in the number of galactose side chains (U. Hiroshi, Trends in Glycoscience and Glycotechnology, 14, 355-376, 2002).

The main use of xyloglucans is in human and animal nutrition. Furthemore, flour obtained by seeds of the above mentioned plants, containing xyloglucans, is commonly used in nutritional industry as a stabilizing, gelling and thickening agent.

The use of xyloglucans in medical devices or as a component in pharmaceutical preparations is also reported, although less frequently. For example, xyloglucans are used as viscosizing agents in products used as lachrymal substitutes, showing a safe toxicological profile even when administered to ophthalmic mucous membrane, which are particularly sensitive to lesions and irritations (M. F. Saettone et al. in U.S. Pat. No. 6,056,950). WO 9729777 (M. Shozo et al.,) discloses a xyloglucan preparation, partially degraded with an enzyme such as galactosidase, for use as an ingredient in the preparation of controlled-release formulations). According to this patent, partial removal of galactose residues from xyloglucan provides high gelling properties, so that an active principle incorporated in such gel can be gradually released in the application site.

It has now been found, and this is the object of the present invention, that preparations of suitably purified xyloglucans combined with appropriate quantities of glycerol show unexpected mucoadhesive properties. The presence of glycerol unexpectedly increases xyloglucan mucoadhesiveness.

Moreover, the glycerol content is directly related to the controlled release behaviour of this xyloglucan preparation, and mucoadhesiveness persists even reducing the viscosity of the solution.

### DESCRIPTION OF THE INVENTION

Object of the invention are mucoadhesive and controlled release formulations consisting of aqueous solutions containing 0.05% to 5% by weight of a natural purified polymer having xyloglucan structure and 10% to 70% by weight of glycerol. These formulations are suitable for the application on human mucous membranes, such as nasal, oral and vaginal mucous membranes, as moisturizing and softening agents or as pharmaceutical release system. The combinations of the invention can be added with active principles and excipients known by those skilled in the art, for the preparation of pharmaceutical preparations or medical devices for human use that are a further object of the invention.

These pharmaceutical preparations and medical devices can he formulated as solutions, gels, ovules, sprays, mouthwashes, creams, ointments, vaginal washes and any other suitable administration systems.

A great number of active principles can be used in the present invention, preferably antibiotics, antimycotics, antiprotozoals, antivirals, antiinflammatories, disinfectants, chemotherapeutics, analgesics, mucolytics, antitussives, decongestants, calcium absorption regulators, hormones and vaccines.

The xyloglucan purified of the present invention is obtained by purification of vegetable extracts of xyloglucans, or commercially available unfinished products with different purity grade for the use in nutritional industry.

The purification is carried out according to the following process. A 0.5% to 5% suspension, preferably 1% to 2%, of raw powder xyloglucan extract is prepared in cold distilled water. The suspension is then poured in four times the volume of boiling distilled water and the solution is kept under boiling for 15 to 30 minutes under stirring. The resulting solution is left to standleft to stand for 10 to 20 hours until the contaminant proteins and fibres precipitate. The solution is then centrifuged at 4000-8000 rpm for a time between 15 and 30 minutes. The supernatant is filtered through a 6 μm polypropylene filter and the filtrate is further centrifuged at 12000-18000 rpm for a time between 30 and 60 minutes. The supernatant can then be sterilized either by filtration through a 0.22 μm polypropylene filter or in autoclave at 121° C. for a time between 20 and 40 minutes. The resulting solution can be directly used as such or lyophilized. The described process affords the purified xyloglucan in a yield between 30% and 80% in respect of the starting product.

Such purification process affords xyloglueans suitable for the pharmaceutical use, in particular providing a safety profile for use in humans, more particularly for the application to mucous membranes. The purification process of the invention provides a product with constant technological properties which guarantee reproducibility in terms of viscosity and mucoadhesiveness. Viscosity is one of more important parameters which characterize a product intended for mucoadhesive preparations. The process of the invention provides a purified xyloglucan with viscosity suitable for the scopes of invention., as determined on a 2% by weight aqueous solution by using a rotational viscosimeter at the temperature of 25° C. operating at a shear rate of 200 sec⁻¹ and a rest time of 15 minutes.

The resulting viscosity values can change depending on the starting raw product. The viscosity values suitable for the use within the scopes of the present invention range between 150 and 800 mPa·sec.

Moreover, the purification process provides a remarkable reduction of protein impurities present in the starting material. Xyloglucans free from protein impurities are necessary as mucous sites are particularly sensitive to irritations. On the other hand, administration to the oral cavity can trigger anaphylactic reactions. For this reason, according to the invention an upper limit of UV absorbance at 280 nm, which is an indirect measure of residual proteins, has been introduced. The purified xyloglucans suitable for the invention should have an absorption value at 280 nm lower than 0.5 abs units when measured in a 2% by weight aqueous solution at the temperature of 25° C.

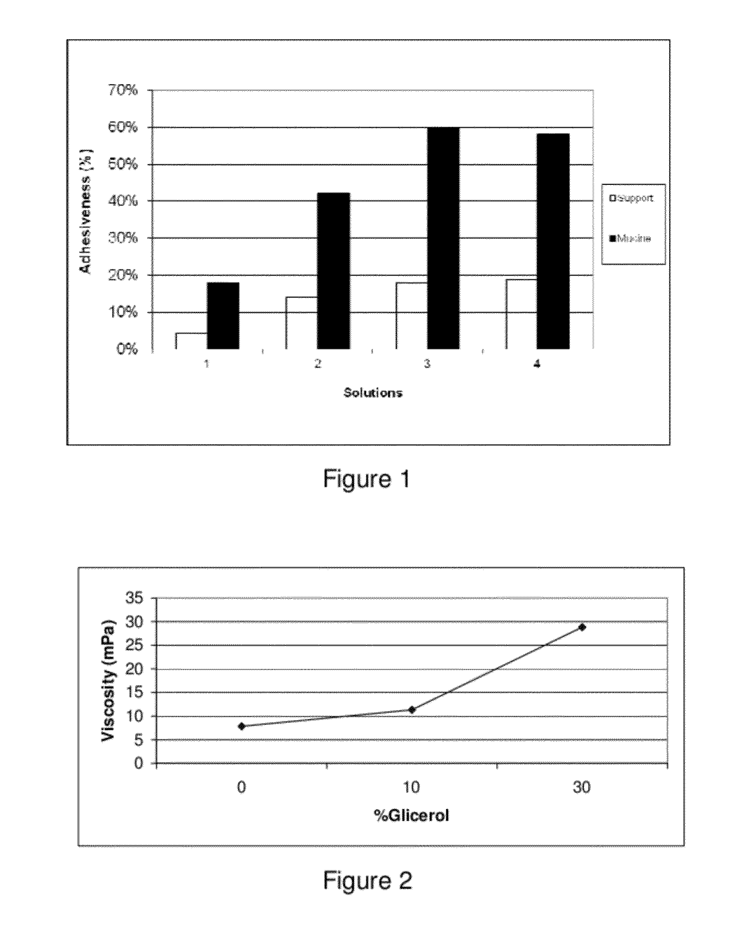

FIG. 1 reports the mucoadhesiveness profile of formulations containing the purified xyloglucan at increasing glycerol concentrations, according to the data from table 2 of example 3,. Such profile shows the synergetic effect of glycerol and xyloglucan in increasing mucoadhesiveness.

FIG. 2 shows that the increase in mucoadhesiveness is not related to viscosity values of the respective solutions, according to the data from table 2 of example 3.

Finally, table 2 of example 3 shows that significant levels of mucoadhesiveness are assured also at low viscosity values, for instance at values lower than 30 mPa·sec.

The latter considerations are particularly relevant in view of the applications considered for the present invention. As a matter of fact, after administration onto mucosa, topical preparations might be subject to two phenomena both having a potentially negative impact on mucoadhesiveness: in fact near the mucous surface, the combined action of temperature and physiological fluids will induce a reduction of viscosity and a dilution effect on the preparation. Conversely, the xyloglucan-glycerol preparation of the invention, maintains mucoadhesiveness at low concentrations and at a level unrelated to viscosity.

According to the invention, the addition of glycerol in concentrations between 10% and 70% by weight to solutions of purified xyloglucan at a concentration between 0.05% and 5% by weight provides a controlled release of drugs in a manner directly related to the glycerol content and unrelated to the viscosity of the final solution.

The controlled release of drug from solutions, prepared as described in Example 2 and reported in Table 1, has been demonstrated by measuring the drug release trough Franz cell over time.

The controlled release property combined with the mucoadhesive property provides the constant, prolonged release of the active ingredient at the site of action, as reported in Table 3 for diclofenac.

The formulation containing xyloglucan and glycerol of the invention, has been evaluated for safety: the obtained results, reported in Examples 11, 12, 13, prove good local tolerability, absence of sensitisation and citotoxicity properties.

The efficacy and safety of the combination of the present invention is also shown by the clinical study reported in Example 10, wherein the use of a medical device in form of vaginal ovule afforded very good results in the treatment of dyspareunia due to vaginal dryness.

The particular mucoadhesiveness together with the controlled release properties of the products of the invention is a determinant characteristic also for the preparation of innovative pharmaceutical forms for drugs release. The advantages are connected to the possibility to keep the pharmaceutical ingredient active at the action site for longer times, increasing the drug bioavailability. Furthermore, the prolonged time of permanence, combined with the drug controlled release, allows to decrease the administration frequency. Such properties can be useful for the administration of drugs to oral, nasal and vaginal mucous membranes.

The present invention also relates to medical devices and pharmaceutical preparations containing, in addition to pharmaceutical active ingredients and excipients, 0.05% to 5% by weight of purified natural polymer having xyloglucan structure and 10% to 70% by weight of glycerol.

According to the present invention, preferred active principles are selected from antiinflammatory agents, such as diclofenac and benzidamine, hormones such as progesterone and calcitonin. Preferred formulation forms are ovules, gels, lavages and sprays.

Examples of pharmaceutical preparations according to the invention are an oropharyngeal spray containing sodium diclofenac, a vaginal gel containing progesterone, a vaginal lavage containing benzidamine hydrochloride and a nasal spray containing calcitonin.

Examples of medical devices according to the invention are a vaginal ovule having hydrating properties and an oropharyngeal spray having softening properties.

Said medical devices and pharmaceutical preparations are described in more detail in the following examples.

### EXAMPLE 1

Preparation of a Purified Xyloglucan

A suspension is prepared by adding 200 ml cold distilled water to 20 g Glyloid® 3S (Dainippon) under powder form. The suspension is poured into 800 ml of boiling distilled water and the solution is kept under boiling for 20 minutes under magnetic stirring. The resulting solution is left to stand for 12 hours, then centrifuged at 5000 rpm for 20 minutes. The supernatant is filtered through a 6 μm polypropylene filter and the resulting solution is further centrifuged at 15000 rpm for 30 minutes. Then the solution is sterilized through a 0.22 μm polypropylene filter. Finally the resulting solution is lyophilized obtaining 14 g of purified xyloglucan preparation.

The viscosity of the purified product at the concentration of 2% by weight in an aqueous solution is 229 mPa·sec, measured at the temperature of 25° C. with a “shear rate” of 200 sec⁻¹ and at a rest time of 15 minutes.

The absorbance of a 2% by weight aqueous solution at the temperature of 25° C., measured at 280 nm, is 0.2 abs units.

### EXAMPLE 2

### Preparation of Solutions Containing Sodium Diclofenac

Sodium methyl-paraoxybenzoate is dissolved in water at the temperature of 70° C., then disodium monobasic phosphonate, sodium diclofenac and glycerol are added to the cooled solution. The solution is kept under stirring until complete dissolution.

Purified xyloglucan is then added and the solution is kept under stirring for 8 hours until complete swelling, then is brought to 100 ml with water.

The compositions of sodium diclofenac solutions are reported in Table 1.

TABLE 1

| Components | Solution 1 | Solution 2 | Solution 3 | Solution 4 |
|---|---|---|---|---|
| Sodium diclofenac | 0.08 | 0.08 | 0.08 | 0.08 |
| (% w/w) | | | | |
| Purified xyloglucan | 0 | 0.4 | 0.4 | 0.4 |
| (% w/w) | | | | |
| Glycerol (% w/w) | 30 | 0 | 10 | 30 |
| Sodium methyl-para- | 0.15 | 0.15 | 0.15 | 0.15 |
| oxybenzoate (% w/w) | | | | |
| Disodium monobasic | 0.22 | 0.22 | 0.22 | 0.22 |
| phosphate (% w/w) | | | | |
| Deionized water | | | | |
| to 100 ml | | | | |

### EXAMPLE 3

### Viscosity and Mucoadhesiveness Measure of Solutions of Sodium Diclofenac

The viscosity of sodium diclofenac solutions prepared according to Example 2 was measured using a rheostat having a measure geometry with a cone plate C 60/1, a “shear rate” of 200 sec⁻¹ and a rest time of 3 minutes.

The in vitro measurement of the mucoadhesiveness of the solutions prepared according to Example 2 was carried out with the method of inclined plane as described by Sandri G. et al. (J. Pharm. Pharmacol., 56, 1083-1090, 2004). The test is carried out by gliding the sample on an inclined inert support covered with a gastric porcine mucin layer. The absorption on the mucin layer is valued referring to the absorption of the same sample on the non-covered inclined inert support.

500 mg of each sample of the solutions reported in Table 1 containing the purified xyloglucan are placed on the higher point of the plane inclined at 60′ and the mixture portion gliding on the plane in a time of 2 minutes is collected and measured by weighing.

The viscosity and mucoadhesiveness values of the sodium diclofenac solutions described in Example 2 are reported in Table 2.

TABLE 2

| | Viscosity | % adhesiveness | % adhesiveness |
|---|---|---|---|
| Solutions | (mPa · sec) | to inert support | to mucin |
| 1 | n.d. | 4.3 | 18.0 |
| 2 | 7.86 | 14.0 | 28.0 |
| 3 | 11.32 | 17.8 | 60.0 |
| 4 | 28.83 | 18.8 | 58.2 |

### EXAMPLE 4

### Evaluation of Controlled Release Properties of a Sodium Diclofenac Solution

The controlled release properties of a solution containing sodium diclofenac prepared according to Example 2, have been evaluated measuring the washability of the viscous solutions on Franz cell.

The solutions, in the equipment thermostated at 37° C., were washed with phosphate buffer at pH 7.4 and the quantity of active ingredient released was measured after 1, 2, 3 and 4 hours. The percentage on the total quantity of released sodium diclofenac is reported in Table 3.

TABLE 3

| | % sodium | % sodium | % sodium |
|---|---|---|---|
| Time | diclofenac | diclofenac | diclofenac |
| (hours) | Solution 2 | Solution 3 | Solution 4 |
| 1 | 31.6 | 26.7 | 6.4 |
| 2 | 46.4 | 35.5 | 14.02 |
| 3 | 48.1 | 38.0 | 24.8 |
| 4 | 52.7 | 38.0 | 28.8 |

### EXAMPLE 5

### Preparation of an Ovule Containing Purified Xyloglucan Having Hydrating Properties

Aloe barbadensis cryodehydrated extract, fish gelatine, sodium methyl p-hydroxybenzoate, sodium ethyl p-hydroxybenzoate and sodium propyl p-hydroxybenzoate are solubilized at 70° C. in 15 ml of water. After complete solubilization, the solution is cooled to 25° C. and the purified xyloglucan is added. The solution is kept under stirring for about 8 hours and after complete swelling is brought to the temperature of 45° C. and glycerol is added.

After homogenisation by mixing, deionized water is added to 30 grams total weight and the product is distributed into 10 moulds.

The amounts of each component for the preparation of 10 ovules are reported in Table 4.

TABLE 4

| Component | Weight | |
|---|---|---|
| Xyloglucan | 300 | mg |
| Aloe barbadensis | 300 | mg |
| Fish gelatine | 600 | mg |
| Glycerol | 10.5 | g |
| Sodium methyl | 9.43 | mg |
| p-hydroxybenzoate | | |
| Sodiumethyl | 4.2 | mg |
| p-hydroxybenzoate | | |
| Sodium propyl | 4.81 | mg |
| p-hydroxybenzoate | | |
| Deionized water to 30 grams | | |

### EXAMPLE 6

### Preparation of a Vaginal gel Containing Progesterone

Methyl parahydroxybenzoate is added at 70° C. in water. After complete solubilization and cooling of the solution, hydrogenated palm oil glycerides of containing the previously solubilized progesterone are added. After complete mixing, purified xyloglucan is added and the mixture it is kept under stirring for 8 hours until complete swelling of the polymer. Lastly glycerol is added and the final solution is stirred.

The amounts of the various components for the preparation of the vaginal gel are reported in Table 5.

TABLE 5

| Component | Weight |
|---|---|
| Xyloglucan | l g |
| Progesterone | 8 g |
| Glycerol | 30 g |
| Hydrogenated palm | 5 g |
| oil glycerides | |
| Sodium methyl | 0.15 g |
| para-hydroxybenzoate | |
| Deionized water to 56 grams | |

### EXAMPLE 7

### Preparation of a Vaginal Lavage Containing Benzidamine Hydrochloride

Trimethylcetylammonium p-toluenesulphonate, rose fragrance and benzidamine hydrochloride are solubilized in water. Purified xyloglucan is added after complete solubilization and the solution is kept under stirring for 8 hours until complete swelling of the polymer. Glycerol is added and the final solution is mixed until complete homogenisation.

The amounts of the various components are reported in Table 6.

TABLE 6

| Component | Weight | |
|---|---|---|
| Xyloglucan | 1 | g |
| Benzidamine hydrochloride | 100 | mg |
| Glycerol | 30 | g |
| Trimethylammonium | 5 | g |
| p-toluenesulphonate | | |
| Rose fragrance | 100 | mg |
| Deionized water to 64 grams | | |

### EXAMPLE 8

### Preparation of a Nasal Spray Containing Calcitonin

Sodium chloride, citric acid monohydrate, sodium citrate dihydrate and hydrochloric acid are added to 65 ml of water. The mixture is kept under stirring until complete dissolution and then 10 ml of water containing the previously solubilized calcitonin are added. The purified xyloglucan is added, keeping stirring for 8 hours until complete swelling of the polymer. Then glycerol is added, the solution is mixed and water is added to the necessary weight.

The amounts of the various components are reported in Table 7.

TABLE 7

| Component | Weight | |
|---|---|---|
| Xyloglucan | 400 | mg |
| Calcitonin | 100.000 | IU |
| 0.1N Hydrochloric acid | 0.15 | mg |
| Sodium chloride | 9 | g |
| Sodium citrate dihydrate | 58 | mg |
| Citric acid monohydrate | 58 | g |
| Glycerol | 15 | g |
| Deionized water to 100 grams | | |

### EXAMPLE 9

### Preparation of an Oropharyngeal Spray Containing Natural Extracts

The natural extracts, potassium sorbate and monobasic sodium phosphate monohydrate are added to 65 ml of water. The purified xyloglucan is added, keeping stirring for 8 hours until complete dissolution. Then water is added to the necessary volume.

The amounts of the various components are reported in table 8.

TABLE 8

| Component | Weight |
|---|---|
| Xyloglucan | 400 mg |
| Erysimum dry extract | 300 mg |
| Althea dry extract | 200 mg |
| Noni dry extract | 300 mg |
| Monobasic sodium | 220 mg |
| phosphate monohydrate | |
| Potassium sorbate | 100 mg |
| Glycerol | 30 g |
| Deionized water to 100 grams | |

### EXAMPLE 10

### Evaluation of the Efficaciousness and Tolerability of Ovules Containing Purified Xyloglucan on Volunteers

The effectiveness and safety of ovules prepared according to Example 4 have been evaluated in the treatment of the dyspareunia caused by vaginal dryness in menopausal women not receving substitutive hormonal therapy.

The goal of the open study was to evaluate the treatment's efficaciousness with the ovules of Example 4 in reducing the symptom train, like dyspareunia, vulvar burning and itch, connected to the vaginal dryness.

8 Women aging between 45 and 65 years in menopause for almost 3 years and without active vaginal infections have been enrolled in the study. The treatment scheme has foreseen the administration of the ovule by vaginal route twice a day for a period of 8 days.

The clinical end point has been evaluated by using a score scale between 0 (absent symptomatology) and 5 (very intense symptomatology).

All patients have completed the treatment. The drug resulted well tolerated and efficacious both at local and systemic level, as shown in Table 9. No side reactions have been observed.

TABLE 9

| | Dyspareunia | | Vulvar burning | | Vulvar itch | |
|---|---|---|---|---|---|---|
| Patient | | End of | | End of | | End of |
| (No.) | Basal | therapy | Basal | therapy | Basal | therapy |
| 1 | 4 | 1 | 5 | 3 | 3 | 1 |
| 2 | 4 | 2 | 4 | 2 | 2 | 0 |
| 3 | 5 | 2 | 4 | 1 | 2 | 0 |
| 4 | 3 | 0 | 4 | 0 | 2 | 0 |
| 5 | 4 | 1 | 4 | 1 | 1 | 1 |
| 6 | 2 | 0 | 3 | 0 | 2 | 0 |
| 7 | 3 | 0 | 2 | 0 | 3 | 1 |
| 8 | 3 | 1 | 3 | 0 | 3 | 0 |
| average | 3.50 | 0.88 | 3.63 | 0.88 | 2.25 | 0.38 |

### EXAMPLE 11

### Local Tolerance Study of a Preparation Containing Xyloglucan and Glycerol

The local effects of the oropharyngeal spray of Example 9, on oral cavity mucous membranes were determined by examining local tissue changes after repeated placement of the test item into the cheek pouch of the Syrian hamster, over a period of 7 days.

One group constituted of 5 male and 5 female animals, was treated daily with the medical preparation at a dose of 0.5 ml for each animal for a total of 3 times in the same day with 2 hours interval, to give a total daily dosage of 1.5 ml for each animal.

Control group animals were treated with sterile water for the same period of time. No indications of a systemic effect of the test were observed during the treatment period. A slight mucous irritation was observed in animals treated with the medical preparation approximately 1 hour after the last application and no reactions were observed 24 hours later, thus proving the good tolerability in the hamster oral cavity mucous membranes.

### EXAMPLE 12

### Delayed Dermal Sensitisation Study of a Preparation Containing Xyloglucan and Glycerol

A Guinea pig model, with the test of Magnusson and Kligman (J. Invest. Dermatol., 268-276, 1969) was used to induce and elicit delayed dermal sensitisation. To induce sensitisation, a test group of 20 animals and a control group of 10 animals were intradermally injected with an emulsion of Freund's complete adjuvant, (anterior sites), the undiluted test item (middle sites) and the test item at a concentration of 50% in an emulsion of Freund's complete adjuvant (posterior sites). One week later this application was boosted by a topical application of the test item at 100% over the injection sites. Control group animals were treated with sterile water for the same period of time. Two weeks after the second induction stage, all animals were challenged by topical application of both the vehicle.

At the end of the study no response was apparent in any animal of the test or control groups and no reaction to the vehicle alone was observed in any animals.

### EXAMPLE 13

### Cell Toxicity Assay of a Solution Containing Xyloglucan and Glycerol

The in vitro cytotoxicity of a solution containing xyloglucan and glycerol has been evaluated on BALB/C3T3 cells and measurement of neutral red uptake for cellular toxicity was performed on cultures of BALB/C3T3 cells treated with different concentrations of the test item and at the end of the treatment, the cultures were examined in order to evaluate changes in the morphology of the cells due to toxicity effects of the test item. Negative and positive control were included in the experiment.

The test item was solubilized in culture medium and concentrations used were 39.1; 78.1; 156; 313; 625; 1250; 2500 and 5000 micrograms/millilitre. Cytotoxicity was analysed after 24 hours treatment time. No change in the morphology of the cells and no relevant reduction in neutral red uptake was observed after treatment with the test item in any experiments at any concentration tested.

1-23. (canceled)

24. A method of administering to a subject an active pharmaceutical ingredient in a mucoadhesive formulation, the method comprising:
identifying a subject in need thereof, and
topically administering to a mucous membrane of the subject a pharmaceutical formulation comprising:
the active pharmaceutical ingredient;
a purified natural xyloglucan at a concentration between 0.05% and 5% by weight of said formulation;
glycerol at a concentration between 10% and 70% by weight of said formulation; and
a pharmaceutically acceptable excipient.

25. The method of claim 24, wherein the formulation is administered to human mucosa.

26. The method of claim 25, wherein the human mucosa is selected from the group consisting of oral mucosa, nasal mucosa, and vaginal mucosa.

27. The method of claim 24, wherein the purified natural xyloglucan has viscosity between 150 mPa·sec and 800 mPa·sec at the temperature of 25° C. operating with a 200 sec⁻¹ shear rate and a rest time of 15 minutes.

28. The method of claim 24, wherein the UV absorbance at 280 nm of a 2% by weight of the formulation of the purified natural xyloglucan at 25° C. is lower than 0.5 abs units.

29. The method of claim 24, wherein the pharmaceutical formulation is in controlled release form.

30. The method of claim 24, wherein the natural xyloglucan is present at a concentration by weight of between 0.1% and 1.8% of said formulation.

31. The method of claim 24, wherein glycerol is present at a concentration by weight of between 15% and 54% of said formulation.

32. The method of claim 24, wherein the active pharmaceutical ingredient is selected from the group consisting of antibiotics, antimycotics, antiprotozoals, antivirals, anti-inflammatories, disinfectants, chemotherapeutics, analgesics, antiseptics, mucolytics, antitussives, decongestants, calcium absorption and regulating agents, hormones and vaccines.

33. The method of claim 32, wherein the anti-inflammatory is diclofenac or benzidamine.

34. The method of claim 24, wherein the formulation is in the form of solutions, gels, ovules, sprays, mouthwashes, creams, ointments, unguents and lavages.

35. The method of claim 24, where in the formulation is in the form of oral solution, or spray, or mouthwash, and wherein the formulation further comprises diclofenac acid or salts thereof, or benzidamine hydrochloride, or a natural extract.

36. The method of claim 24, in a form selected from the group consisting of a vaginal gel further comprising progesterone, a vaginal lavage further comprising benzidamine hydrochloride, and a nasal spray further comprising calcitonin.

37. The method of claim 24, wherein the natural xyloglucan is obtained from a source selected from the group consisting of Tamarind seeds, Detarium senegalense J. Gmelin, and Afzelia africana.

38. The method of claim 24, wherein the subject in need thereof has mucous dryness.

39. A method of administering to a subject an active pharmaceutical ingredient in a mucoadhesive formulation, the method comprising:
identifying a subject in need thereof, and
topically administering to a mucous membrane of the subject a pharmaceutical formulation comprising:
the active pharmaceutical ingredient;
a natural xyloglucan at a concentration between 0.1% and 1.8% by weight of said formulation;
glycerol at a concentration between 15% and 54% by weight of said formulation; and
a pharmaceutically acceptable excipient.

40. The method of claim 39, wherein the active pharmaceutical ingredient is an antibiotic, antimycotics, antiprotozal, hormone, antiviral, anti-inflammatory, disinfectant, chemotherapeutic, analgesic, mucolytic, antitussive, decongestant, calcium absorption regulator, hormone or vaccine.

41. The method of claim 40, wherein the anti-inflammatory is diclofenac or benzidamine.

42. The method of claim 39, wherein the natural xyloglucan is obtained from a source selected from the group consisting of Tamarind seeds, Detarium senegalense J. Gmelin, and Afzelia africana.
